(19) United States
(12) Patent Application Publication
(10) Pub. No.: US 2012/0238976 A1
(43) Pub. Date: Sep. 20, 2012

(54) Male urine collection and storage device

(76) Inventor: James B. T. Foster, New York, NY (US)

(21) Appl. No.: 13/065,251

(22) Filed: Mar. 17, 2011

(51) Int. Cl. A61F 5/453

(52) U.S. Cl. 604/353

(57) ABSTRACT

A urine collection system and storage devices adapted for a male which can be worn under clothes during typical daily activities. The system includes a device having a having a modified condom-collector for attachment to a penis leading into a detachable reservoir, as well as an application sheath and sizers for fitting an appropriately sized collector for a user.

### FIELD OF INVENTION

This invention is related to the art of urine collection devices, and is more particularly related to urine collection devices for males which can be worn under clothes during typical daily activities.

### BACKGROUND OF THE INVENTION

Acquired urinary incontinence in the adult male is most often a consequence of trauma, either accidental or as a complication of the surgical or radiological treatment of prostatic disease. Paraplegia often accompanies accidental trauma, and its special problems require special solution. This invention is intended for the relief of ambulant men who are often discouraged by fear or embarrassment from facing the outside world.

A man produces about 1 c.c. (or 20 drops) of urine per minutes, or about 2 ounces per hour. During an eight hour period in which a man may be in bed, adequate relief can be secured with special garments and pads, but in the sixteen hours during which a man may be awake he may be compelled to release up to a quart of urine in various settings.

Not surprisingly, this problem has attracted attention and inspired invention. In a prior system, a favored expedient has been a tripartite system consisting of a collector, a conduit and a storage receptacle or reservoir. The collectors, often based on a preconception of “one size fits all” tend to be complicated and cumbersome. The conduits, usually tubular and having limited flexibility, tend to be awkward. The reservoirs, usually worn on the thigh but sometimes on the lower leg, tend to be bulky, noticeable and difficult to empty.

Accordingly, there is a need for an improved urine storage device. More particularly, there is a need for an improved urine storage device for males which can be worn under clothes during typical daily activities.

### OBJECT OF THE INVENTION

It is an object of the invention to provide a urine collection and storage device which addresses the aforementioned problems.

It is an object of the invention to provide a urine collection system having a reservoir which can be readily emptied by a user. It is a further object of the invention to provide a urine collection system having a reservoir which can be readily disconnected from a conduit by a user for emptying the reservoir, and which can be easily reconnected.

It is an object of the invention to provide a urine collection system having a collector which can fit snugly onto a user's penis which can collect and distribute urine via a conduit through a connector to a reservoir, without leakage, pooling or backflow of urine proximate to the reservoir.

It is an object of the invention to provide a urine collection system utilizing commonly available components.

It is an object of the invention to provide a urine collection system which can be discreetly utilized by a user under his clothes in either a standing or sitting position.

It is an object of the invention to provide a urine collection system which can be readily modified and adapted for use by different users.

It is an object of the invention to provide a urine collection system having a plurality of parts which can be readily replaced and/or cleansed.

It is an object of the invention to provide a urine collection system having a choice of parts and connections which are easily usable by older gentlemen discreetly.

### SUMMARY OF THE INVENTION

A urine collection device according to the invention is provided with a collector, a conduit, a reservoir, a flow restrictor and at least one leg strap. The collector is connected to the conduit for passing fluid to the conduit, and the conduit is connected to the reservoir for passing fluid to the reservoir. In addition, the leg strap is connected to the reservoir for attaching it to a user's leg. The collector is made from a flexible material to provide a substantially water-tight fit over a user's penis. In one embodiment, the conduit is a tube having a smaller diameter than the collector. In addition, a restrictor is provided which can be a small aperture whereby fluid from the conduit can pass slowly into the reservoir, and whereby rapid backflow of fluid from the reservoir is avoided.

In an alternative embodiment, the collector and conduit can be formed as an integral part and can be fabricated from the same flexible material.

In another embodiment, the reservoir can be a chamber formed from a flexible material, such as rubber, and is provided with an attachment point for receiving the connector. In addition the reservoir can have a number of attachment points for connecting the leg straps. For example, the attachment points can be loops and associated hooks.

In a further embodiment, the reservoir can have an inlet that is offset from the center of a side of the reservoir and is near the end of a top side of the reservoir. In addition, the reservoir can have a curved inside face to improve comfort when worn by a user.

A cinch can also be provided over the conduit for securing the conduit on the connector. For example, a tube passing through the connector can be provided with a notch at an outside end of the tube and around at least a portion of the circumference of the tube for receiving a portion of the conduit being secured by the cinch.

In alternative embodiments, the collector and conduit are provided as having a similar diameter, and can be provided as a unitary structure or separate structures.

Furthermore, the connector can be provided with a tube passing through it and have a screw cap adapted to connect to a top edge of the reservoir.

In another embodiment, the flow restrictor can be a rubber cot having a small hole at a lower end, and be located at an inside end of the tube.

Alternatively, the flow restrictor can have an distal restrictor with an aperture at an its distal end. In addition, the flow constrictor can have a swagged chamber, a check valve, a ball obturator, a disciform obturator and/or obturator housing.

In a further concept of a system according to the invention, the urine collector includes a collector sizer.

### BRIEF DESCRIPTION OF THE DRAWINGS

Preferred embodiments of the inventive devices are illustrated in the drawings and are described below, though not necessarily to scale.

FIG. 1 illustrates a front and perspective view of an embodiment of a urine collection device according to the invention, as can be positioned on a user's body, partially shown.

FIG. 2 illustrates a front and perspective view of an embodiment of a collector and conduit for a urine collection device.

FIG. 3A illustrates a front and perspective view of an embodiment of a reservoir for a urine collection device.

FIG. 3B illustrates top, side and perspective views of an embodiment of a leg strap for a urine collection device.

FIG. 3C illustrates a side view of an embodiment of a reservoir for a urine collection device.

FIG. 3D illustrates a side view of an embodiment of a reservoir for a urine collection device.

FIG. 4A illustrates a side and perspective view of an embodiment of a urine collection device according to the invention, as can be positioned on a user's body, partially shown.

FIG. 4B illustrates a front and perspective view of an embodiment of a urine collection device according to the invention, as can be positioned on a user's body, partially shown.

FIG. 4C illustrates a cross-section view of an embodiment of a reservoir for a urine collection device according to the invention, as can be positioned on a user's leg, partially shown in cross-section.

FIG. 4D illustrates a front and perspective view of an embodiment of a urine collection device according to the invention, as can be positioned on a user's body, partially shown.

FIG. 4E illustrates a cross-section view of an embodiment of a reservoir for a urine collection device according to the invention, as can be positioned on a user's leg, partially shown in cross-section.

FIG. 5A illustrates a front and perspective view of an embodiment of a collector, conduit and connector for a urine collection device.

FIG. 5B illustrates a front and perspective view of an embodiment of a connector for a urine collection device.

FIG. 5C illustrates a front and perspective view of an embodiment of a connector and a reservoir, partially shown, for a urine collection device.

FIGS. 6A-H illustrate front and cross-section views of embodiments of a connector for a urine collection device.

FIGS. 7A-C illustrate front and cross-section views of embodiments of a connector for a urine collection device.

FIGS. 8A-D illustrate front and cross-section views of embodiments of a connector for a urine collection device.

FIGS. 9A-C illustrate perspective, side and top views of embodiments of a connector sheath for a urine collection device.

FIGS. 9D illustrates a cross-section view of embodiments of a connector sheath for a urine collection device.

FIGS. 9E illustrates cross-section views of embodiments of a connector sheath for a urine collection device.

FIG. 10A illustrates perspective views of several embodiments of a collector sizer for a urine collection device.

FIG. 10B illustrates a perspective view of an embodiment of a collector sizer for a urine collection device.

FIGS. 11A illustrates a perspective view of an embodiment of a collector and conduit for a urine collection device.

FIGS. 11B illustrates a perspective view of an embodiment of a collector and conduit for a urine collection device.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION

A detailed description of a urine collection and storage device according to the invention is provided below for general applicability. In addition, several specific embodiments are provided as examples of the devices which one of ordinary skill in the art may apply these teachings to address specific problems and to illustrate the benefits and improvements of the device over known solutions.

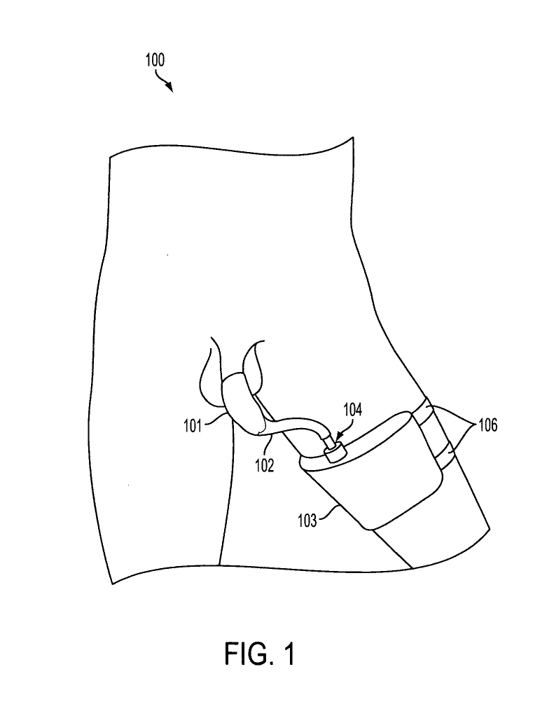

A system 100 for controlling and containing urinary flow is shown in FIG. 1. A urine collection and storage system 100 according to the invention comprises a collector 101, a conduit 102, and a reservoir 103. The collector 101 forms a substantially water-tight connection with a user's penis, and is connected to a conduit 102 for transferring collected urine and other material to the reservoir 103. The conduit can lead into an inlet in the reservoir via a connector 104 which can be incorporated in and/or integral to a removable closure of a reservoir, such as a screw cap. The reservoir is adapted to be comfortable to wear and easy to empty.

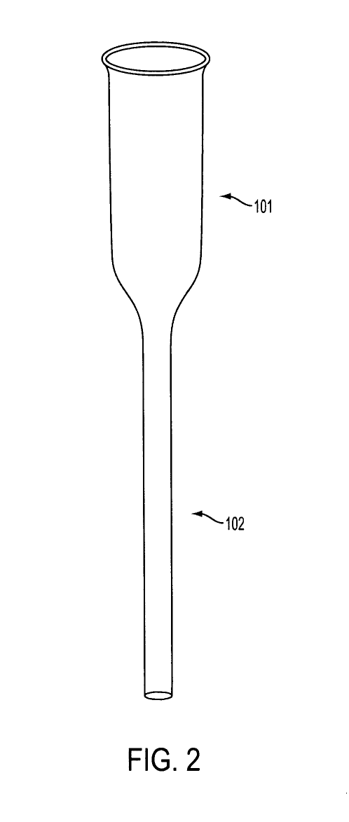

FIG. 2 shows an embodiment of a collector 101 and conduit 102 in accordance with the invention. In one embodiment, the collector 101 is formed from the same material as the conduit 102 and is continuous therewith. For example, the collector 101 and conduit 102 can be fabricated from a condom-like tube of latex or some like substance. It is preferably sized snugly to accommodate the penis and the inlet-closure respectively. Transition from proximal to distal diameters, or from the collector 101 to the conduit 102, can be accomplished by shouldering or tapering or, alternatively, by bunching or folding and securing with an elastic cincture. The collector is preferably provided with sufficient elasticity to permit a very small number of proximal sizes to accommodate a number of penile diameters. The material of the collector 101 and conduit 102 preferably has sufficient flexibility, expandability, and toughness to accommodate a wearer's typical movements without impairing his or the device's function.

In an alternative embodiment, the collector 101 can be a condom to provide a water-tight seal over a penis. The condom can have its tip cut out to receive the conduit 102 which can be a rubber tube. The collector 101 should be sized to the subject's flaccid penis to thereby ensure retention and water-tightness of the device. In a modified condom, the diameter of the proximal part can vary to conform with the subject's penile diameter. The distal part should conform to the diameter of the connector tube in the screw cap.

This tube in the screw cap could be focally-waisted to facilitate securing with a rubber band. In alternative embodiments, further security can be provided wherein the collector 101 can include a cushion material, such as bubble wrap, to be wrapped around the shaft of the penis after the collector 101 has been attached. The cushion material can be secured around the collector 101 with a strap such as a fabric hook-and-loop fastener.

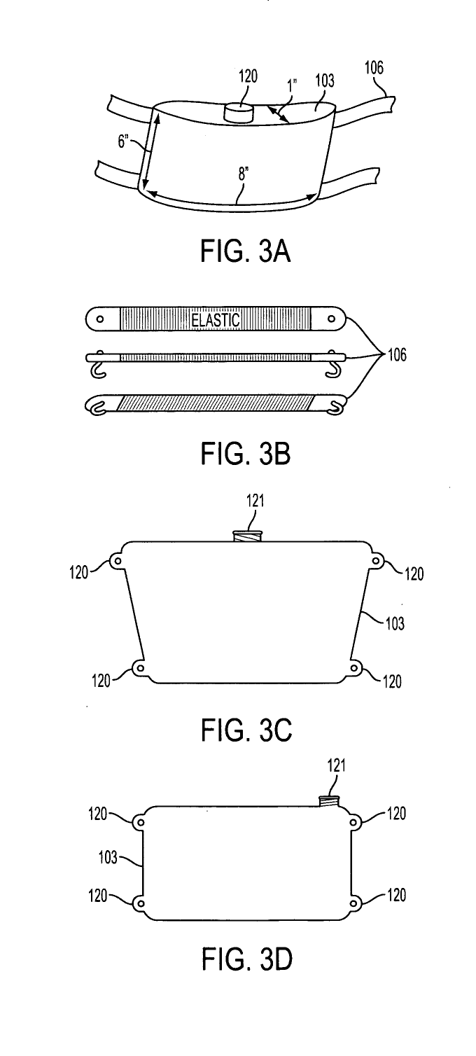

FIG. 3A shows an embodiment of a reservoir 103 in accordance with the invention. The reservoir 103 is a container having at least one opening 121 and at least one attachment point 120 for securing the reservoir to a user's leg. The reservoir 103 can be a rectangular or a shallow truncated pyramidal bag. The reservoir is preferably formed of a flexible material such as rubber to conform to the size and shape of a user's leg. It can be appreciated that the size of the reservoir can vary. The reservoir can be provided in the shape of a box, or it can have one or more sides tapered which can provide comfort for the user. In one embodiment, a satisfactory size for the container is 10″×6″×1″, which for a rectangular bag, could give a capacity of 900 cc or, in a tapered bag, perhaps 750 cc, which in the latter case, would allow 12 hour filling. A 6-hour interval could fill half. In addition, leg straps 106 are provided, for attachment of the reservoir to a user's leg, as described more fully below. It can be appreciated that the reservoir can be adapted for use by either leg.

FIG. 3B shows an embodiment of a leg strap 106 in accordance with the invention. One or more leg straps 106 are provided to secure the reservoir to a user's leg at an attachment point 120. It can be appreciated that a leg strap can be a band, a strap, or other length of material, such as a strap having a fabric hook-and-loop fastener, which can be attached to the reservoir and can readily connect and disconnect the reservoir to or from the user's leg. In addition, a leg strap 106 can be made to be adjustable, such as providing the strap with a buckle through which a length of the strap 106 can be passed to adjust its length. In one embodiment, a leg strap 106 is provided as an elastic material whereby a hook connector can be used at either end of the leg strap to connect the leg strap 106 to the reservoir 103.

For example, as shown in FIGS. 3C and 3D, a reservoir 103 can be provided with attachment points 120, such as four tags, each with a grommet, at the upper and lower edges of each side. Two straps, upper and lower, fixed in these grommets can permit the bag to be placed over the front of the thigh and secured behind.

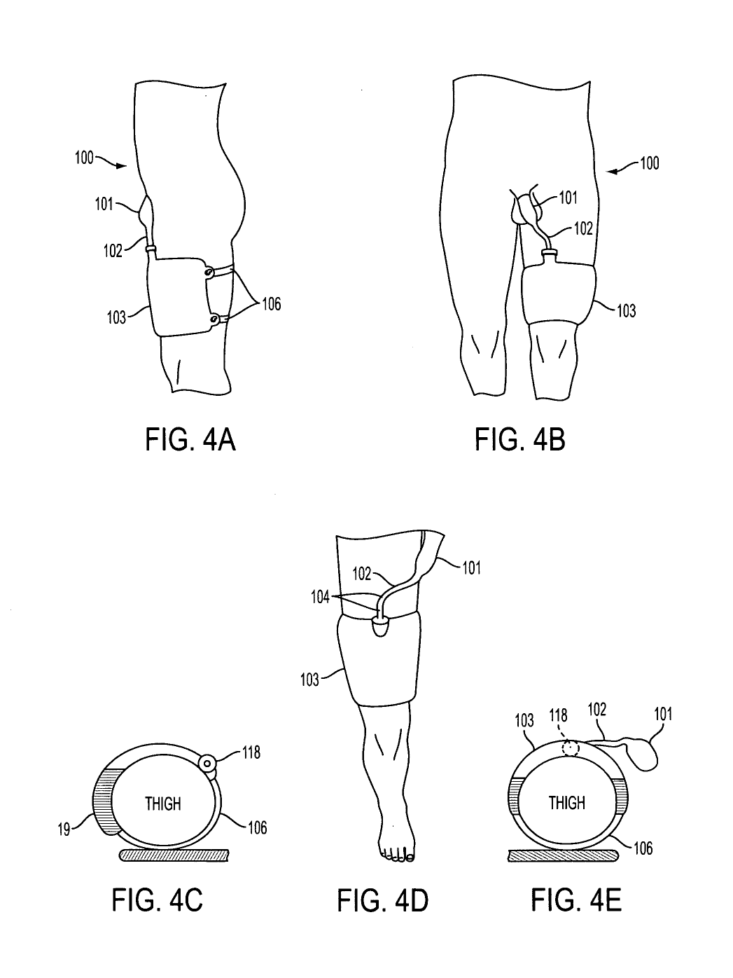

The reservoir is provided with at least one opening 121 through which fluid can be passed into and stored by the reservoir 103. As shown in FIGS. 3C and 3D, the opening can be provided at a top side of the reservoir 103, and can be position either centrally or offset from the center of a side of the reservoir 103. In an embodiment wherein the opening is approximately in the center, either the right or left thigh could be utilized. However, where the opening is provided off to the side, it is preferable to connect the reservoir to in a position at a front end and center in the thigh. In either case, but more especially in the latter case of the opening off to the side, when a subject is sitting and his thigh horizontal, gravity would promote the contained urine to remain away from the inlet, thereby obviating backflow, as illustrated in the cross-section view of FIGS. 4A-E. In either case, the container would not be subject to backflow pressure.

In one embodiment, the reservoir could be fashioned in a manner similar to a hot-water bottle. Indeed, in an embodiment utilizing commonly available materials, a modified rubber hot water bottle could be used. In one embodiment, such as shown in FIGS. 3C and 3D, a reservoir can be provided with tabs having holes which would enable it to be secured with leg straps 104 fashioned from cords to be tied, or elastic straps with hooks at either end. The opening 121 can be provided as a screw top inlet offset from the center and near one end, so that the wearer could dress the device 100 to the left or right as preferred.

FIGS. 4A-E show two embodiments of the urine collection devices in several views. Specifically, FIG. 4A shows a left hand side view of a user employing an embodiment of a urine collection device wherein the reservoir inlet 118 is offset from the center of the reservoir 103. FIG. 4B shows a front side view of this embodiment. FIG. 4C shows a cross-sectional view of the left leg with the user in a sitting position wherein an embodiment of the device 100 has been adapted for use with the left leg wherein the reservoir inlet 118 is offset from the center of the reservoir 103. The reservoir inlet 118 can be positioned such that it is just below the uppermost portion of the thigh when the user is in a sitting position and this fluid that is collected while the user is in a sitting position tends to flow over the thigh and into a portion of the reservoir away from the inlet. Accordingly, FIG. 4C shows that the fluid is maintained in the reservoir 103 while is a horizontal position, maintains the fluid in a portion of the reservoir away from the reservoir inlet 118, thereby preventing backing up of the fluid into the conduit 102 and/or collector 101.

FIG. 4D shows a front side view of a user employing an embodiment of a urine collection device wherein the reservoir inlet 118 is in a center portion of the reservoir 103. FIG. 4E shows a cross-sectional view of the right leg with the user in a sitting position wherein an embodiment of the device 100 has a reservoir inlet 118 in a center portion of the reservoir 103. Accordingly, FIG. 4E shows that the fluid is maintained in the reservoir 103 while is a horizontal position, and the reservoir maintains the fluid in a portion of the reservoir away from the reservoir inlet 118, thereby preventing backing up of the fluid into the conduit 102 and/or collector 101.

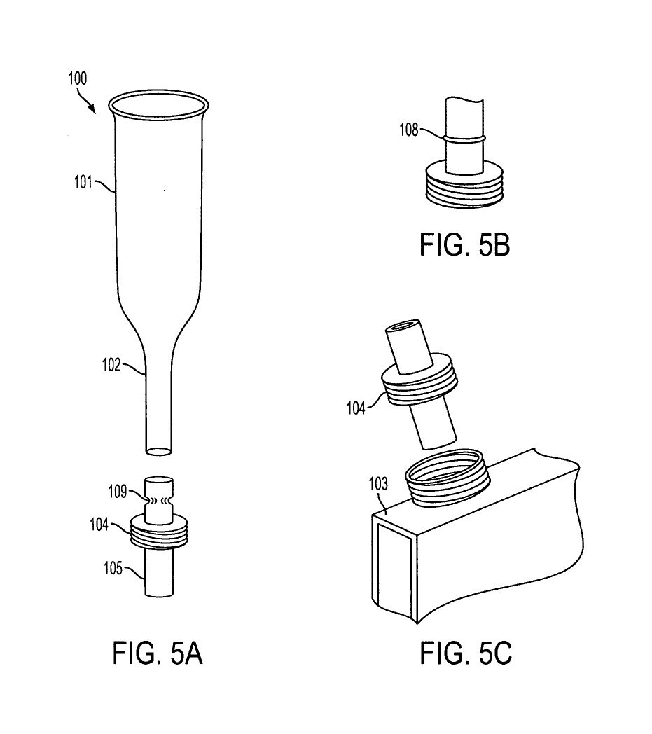

FIG. 5A shows portions of an embodiment of a urine collection device 100 according to the invention, including a collector 101, a conduit 102, and a connector 104 to a reservoir 103 (not shown). The connector 104 includes a cap which can be screwed or snapped into or over a receiving portion of a reservoir inlet 118 (not shown) which can be provided with a corresponding screw or snap connection for fixedly receiving the connector 104. In one embodiment, the connector 104 is provided with a tube which connects to the conduit 102 and transfers fluid to the reservoir 103. The conduit 102 can be connected to the connector 104 by slipping a flexible end of the conduit 102 over the tube. For example, in one embodiment the closure comprises a shallow cylindrical screw-cap in whose center is a perpendicular tube which extends above and below the screw cap.

In one embodiment, the diameter of the tube is preferably uniform but can include a shallow, waisted zone, or notch 109 at a portion of the tube. The notch can be provided to accommodate an elastic cincture, or cinch 108, to ensure tight fit over a distal end of the conduit 102 where it overlaps with the tube of the connector 104. In one embodiment, the cinch 108 is a rubber torus to attach conduit to inlet-closure, but can be fabricated from stiffer material such as plastic or metal and provided as a c-shaped shaped clamp. It can be appreciated that a cinch prevents leakage, and accordingly a cincture that completely surrounds the conduit may be preferable depending on the materials chosen. One advantage of the invention is that the container can be easily removed temporarily to facilitate emptying in a booth in a men's room. Either the notch or the cinch 108 or both can be included to prevent the conduit 102 from accidentally becoming disconnected from the connector 104. In addition, use of a notch and/or cinch facilitates connection of the conduit 102 to the connector 104. In one embodiment, either the connector 104 or the inlet, or both, can be made of metal or plastic or a combination thereof.

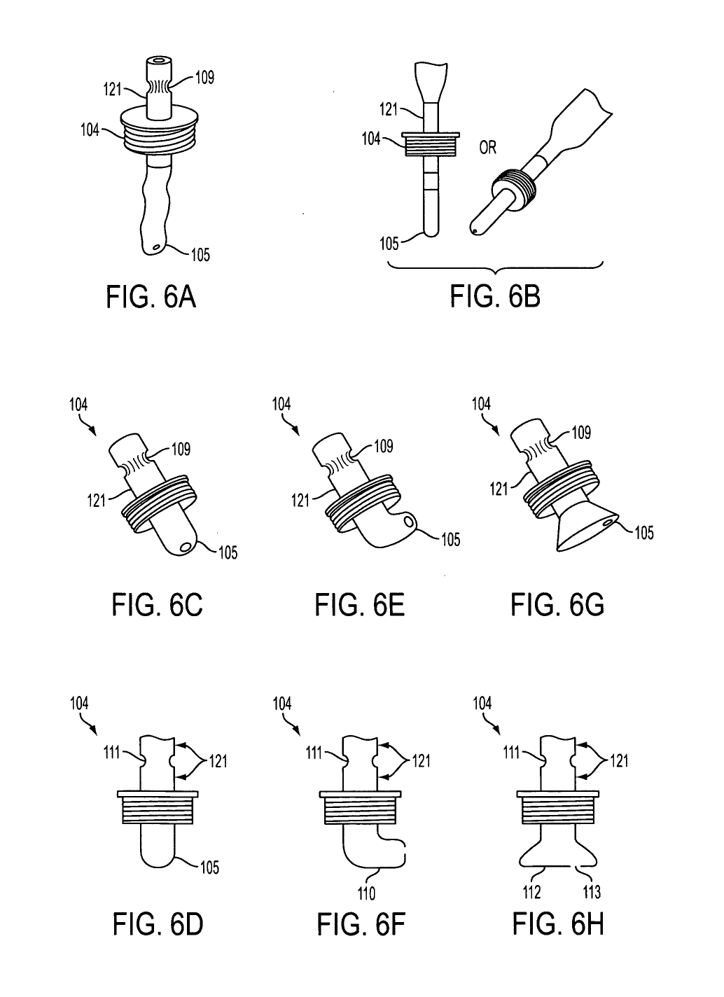

As shown in FIG. 5A and FIG. 6A-6B, the connector 104 can include an inlet closure unit or flow restrictor 105 that facilitates the passage of fluid into the reservoir 103 and discourages reflux (i.e., backflow) of urine from the reservoir 103 back up into the conduit 102. In one embodiment, the flow restrictor 105 is provided with a small hole to permit the flow of urine into the reservoir 103. For example, in one embodiment, the flow restrictor 105 can be formed of flexible material, such as a small condom or rubber cot, which can fit tightly over the inside end of the connector tube which protrudes into the reservoir. The condom or cot is provided with a small hole at its tip to permit the flow of urine, into the reservoir 103. An object of the embodiment of a device having a flow restrictor as shown in FIGS. 6A-B is to allow urine to flow by gravity into the container which is fastened around the thigh, and to prevent backflow if the wearer sits or raises his thigh. In an alternative tube for the inlet, the upper portion of the connector tube 121 includes a notch 109 or a waisted portion 111 to facilitate securing of the condom's lower end with a cinch 108, such as a rubber band, clip or torus.

FIGS. 6C-6F show three alternative embodiments of a connector 104, with FIGS. 6C, E, G showing perspective views of each embodiment, and FIGS. 6D, F, H showing side, cross-sectional views of the embodiment shown in the above Figure. The connector 104 can include a connector tube 121 which provides for fluid flow through the connector and provides for a connection to the conduit 102. On an inside portion of the connector tube 121, its distal end can be closed by including various forms of flow restrictors 105. For example, as shown in FIGS. 6C-6D, the flow restrictor 105 can be a miniature condom having a small central aperture.

In an alternative embodiment, as shown in FIGS. 6E-6F, the flow restrictor 105 can include a distal restrictor 110 which can be formed as a tube with a bent or perpendicular end at a distal end. The bent or perpendicular end can provide for a redirection of the flow of fluid exiting the connector 104, such as to an area of the reservoir 103 away from where the fluid may collect. For example, in one embodiment, the tube is bent so as to be disposed with its outlet facing an anterior direction relative the user, such as outward and away from the surface of the user's thigh. In addition, or in the alternative, the tube is bent so as to be disposed with its outlet facing in an upward direction relative the user. In addition, or in the alternative embodiment where the reservoir is formed so as to collect fluid at an outside area of the user's thigh, the tube can be bent so as to be disposed with its outlet facing in toward the inseam of the user.

In one embodiment wherein the connector 104 includes a screw cap for connection to the reservoir, the bottom of the tube in the screw-capped inlet is curved to obviate or minimize reflux where the thigh is horizontal, such as in a sitting position. The flow restrictor 105 is preferably formed of a material that can retain its shape, such as plastic or metal. For example, in one embodiment having a curved restrictor 110, the restrictor can be curved upward, anteriorly, or turned away from a user's thigh, so that when a user is sitting the aperture to the distal restrictor 110 is prevented from becoming submerged within fluid that has pooled. Similarly, a swagged restrictor 112 can be provided to this effect with the additional benefit to reducing the potential to pierce or damage the inside surface of the reservoir 103.

In an alternative embodiment, as shown in FIGS. 6G-6H, the flow restrictor 105 can include a swagged restrictor 112 which provides for a redirection of the flow of fluid exiting the connector 104, such as to an anterior area of the reservoir 103. The swagged restrictor 112 includes a splayed portion which creates a space within a reservoir 103 made of flexible material and ensures that a the walls of a reservoir made of flexible material do no collapse in an area of the exit 113 of the restrictor 112, thereby interfering with the flow of fluid and thus prevents fluid from backing up into the conduit 102. The swagged restrictor is preferably formed of a material that can retain its shape, such as plastic or metal, and can be provided with an exit aperture near its outer edge.

In the embodiments shown in FIGS. 6E-H, the exit aperture 113 should be situated in an anterior position or direction, such as when an embodiment of a connector 104 includes a screw cap, so that when the thigh is horizontal the aperture is above the fluid level. Thus, an object of any of the above variations is to facilitate the downward passage of urine when the subject is standing, and to minimize or prevent the reflux of fluid when the user is seated or raising his thigh. Regarding the four above variations of inlets, two factors should be borne in mind. First, as the reservoir fills, diminishing air volume in this closed system can inhibit fluid passage in the same way that a fingertip can control fluid release from a laboratory pipette. Second, since the reservoir is draped over the front of the thigh, when the subject sits, contained fluid can flow to both sides away from the central inlet. Thus, the lower portion can be swaged on and positioned so that the exit hole is uppermost when the thigh is horizontal.

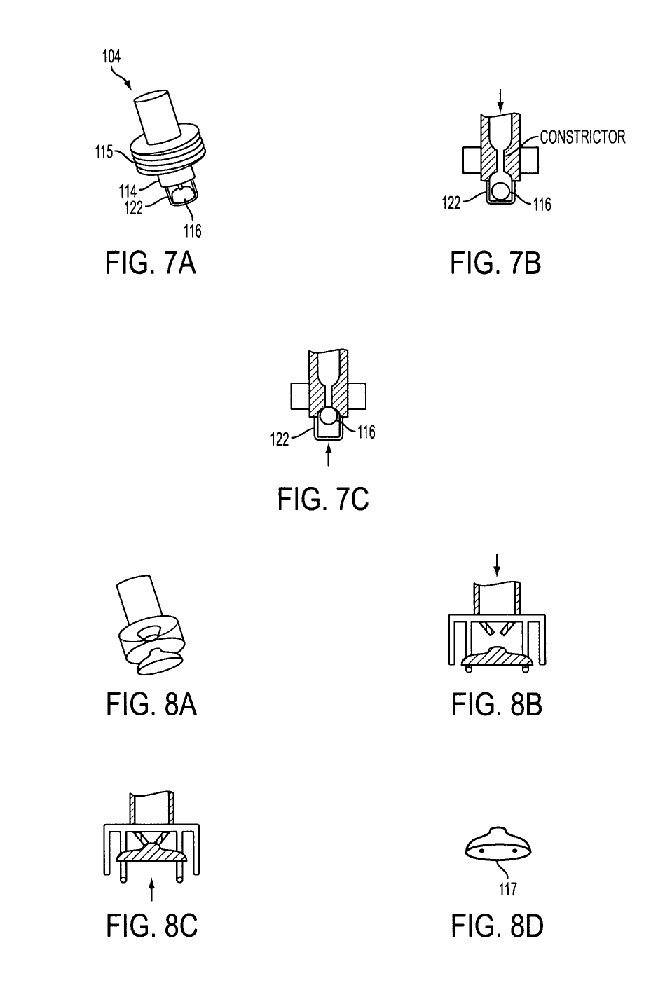

FIG. 7 shows an alternative embodiment of a connector 104 according to the invention. FIG. 7A shows a perspective view of the connector 104, and FIGS. 7B and 7C show side, cross-sectional views. Check valves to direct urinary flow into the container and prevent backflow. The check valves can be incorporated into the screwcap. One embodiment uses a ball-bearing as a ball obturator 116. In addition, an obturator housing 123 can be included to contain the ball obturator 116.

FIG. 8 shows an alternative embodiment of a connector 104 according to the invention. FIG. 8A shows a perspective view of the connector 104, and FIGS. 8B and 8C show side, cross-sectional views. FIG. 8D shows a detailed view of a disciform obturator 117. In addition, an obturator housing 123 can be included to contain the disciform obturator 117.

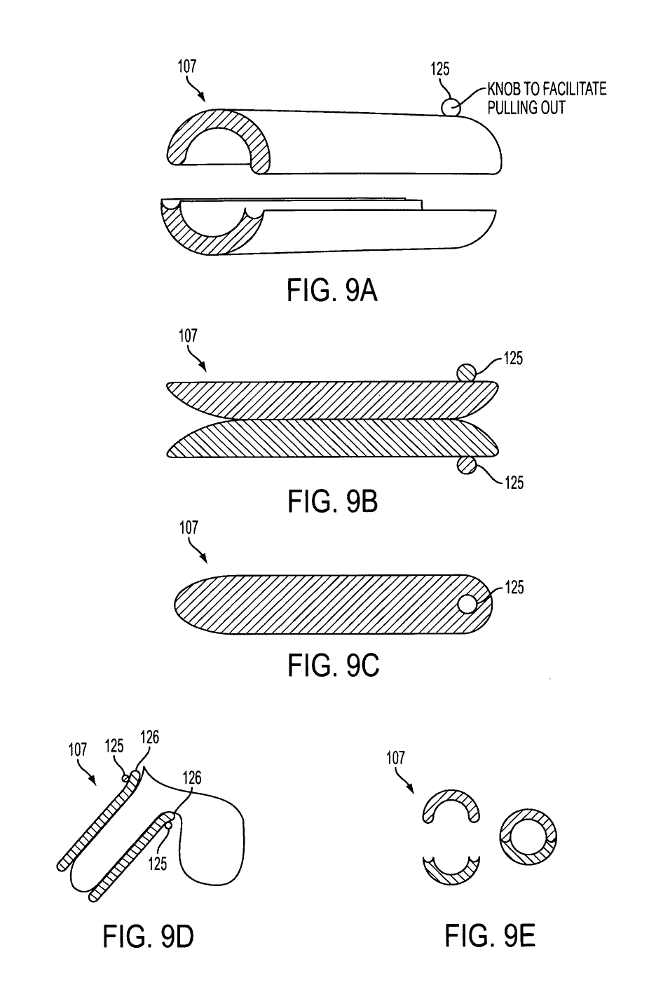

FIG. 9 shows a several views of penile application sheath 107 for use with a urine collection device 100 according to the invention. FIGS. 9A-C illustrate perspective, side and top views of embodiments of an application sheath (i.e., adapter) designed to facilitate placement of a urine collection device on a flaccid penis. FIG. 9D illustrates a longitudinal cross section of an adapter in place on a penis. FIG. 9E illustrates transverse cross-sections of the two halves of the application sheath, separately and opposed.

The penile application sheath 107 is provided to facilitate placement of the proximal part of the device 100 upon a flaccid penis. Most incontinent men are also impotent, whether because of aging or as a sequel to surgical, radiological or hormonal treatment of prostatic disease. A penile application sheath 107 can be provided in an appropriate-size to effect proper placement and retention of the collector 101. A user can insert each of the two halves of the application sheath 107 in the proximal opening of the collector 101, which is preferably done after the user has applied each side of the sheath over his penis. After the collector 101 has been extended over the sheath 107, the user can remove each half of the sheath 107. Preferably, the collector 101 is fabricated of material that is flexible enough to stretch around the sheath and retain sufficient elasticity to permit a snug fit around the user's penis once the sheath is removed.

The penile application sheath 107 can be formed in at least two parts, preferably two smooth, hemi-cylindrically-curved equal lengths which when joined can form the shape of a tube. The sheath parts can be fabricated from thin-walled, rigid or semi-rigid material, such as plastic or metallic stock, and can be provided with joining edges, one of which can be curved up from the convex surface at one end, and semicircular at the other, such as shown in FIG. 9. The long edges of a first half, which can be designated as an upper half, can be provided as convexly round. The long edges of the second, or lower half, can be provided as concavely round, so that apposition of the long edges of both the upper and lower portions can connect and form a stable tubular structure. A preferred length can be from 4-6″, and a preferred diameter can be from 1-2″. If penis and collector are congruently sized there should be no slippage since the length and flexibility of the condom-collector should preclude tension. In addition, the proximal end can be flat with a centrally placed terminal or sub-terminal projection 125, as shown in FIGS. 9A-C, which can facilitate withdrawal or alternatively, can a curved or flared portion 126 at its proximal end which can also facilitate removal and provide additional comfort when used.

A closer fit can be obtained by encircling the covered penis with a length of bubble-wrap secured midway with a wide, not-too-tight rubberband. The proximal end of the device can have a diameter to vary according to that of the subject's flaccid penis. The diameter of the distal portion can be uniform, to conform to the diameter of the proximal end of the inlet-closure. This end may be cut, to obtain the length giving the best fit. The same principle can be utilized to make a transparent device in several diameters properly to size the penis in order to choose the properly-sized collector-conduit in order to secure a snug fit.

The penile application sheath 107 or adapter can be used in the following manner. The subject places the two halves of the adapter around the penis, forming a stable, cylindrical, rigid unit whose rounded distal ends facilitate drawing the proximal end of the urine collection device while the cylinder is being grasped proximally between the thumb and forefinger. Thereafter a user can take the covered cylinder by his other hand, direct his penis downward, and withdraw the upper half of the adapter. Thereafter, the user can direct the penis upward and withdraw the lower half of the adapter. At the conclusion, the penis is firmly and comfortably enveloped by the proximal portion of he collector.

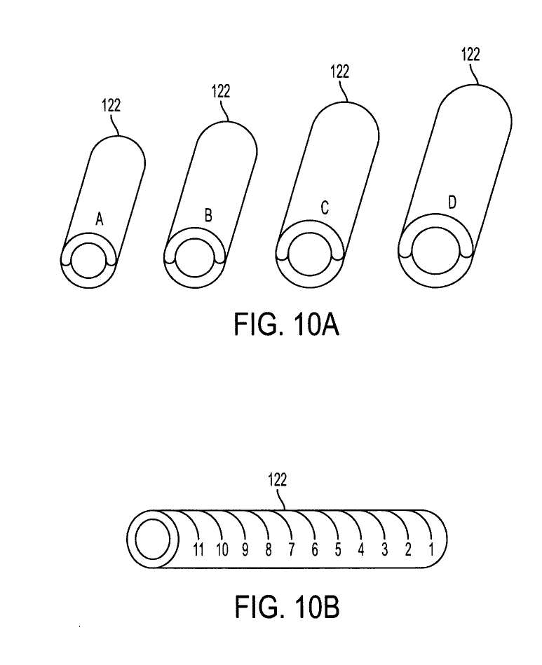

As shown in FIGS. 10A-B, an alternative embodiment of a system according to the invention includes a collector sizer 122 which can provide proper measurement for a collector 101 to ensure a snug, durable, and water-tight fit of the collector 101 to the flaccid penis. In addition, several collector sizers 122 of varying sizes can be provided to provide a measurement of the diameter of the user's penis in order to choose the best sized collector 101 and thereby ensure proper fit by skilled individual, such as a physician, without undue discomfort or embarrassment to the user being measured. Similar to the penile application sheath 107, the collector sizer 122 comprises two hemicylindrical halves of a tube, divided length-wise, which when coupled approximately form the shape of a cylindrical tube. The adjoining edges of the two halves can be shaped so that the two halves can only be joined in one direction, thereby ensure proper use of the sizer 122 and provide for greater stability. For example, the long edges of one side can be convex, and the other side concave. In one embodiment of the sizer 122, the sizer 122 comprises two hemicylindrical parts which together form a tube, at least one of which is transparent and includes markings for length measurement down the length of the sizer 122. In addition, or in the alternative, several sizers 122 having different diameters can be provided. In either case, a portion of each sheath 107 can also be marked with an arbitrary designation, such as a letter A-Z, corresponding to a condom having a corresponding diameter. For example, a physician can fit a sizer 122 around the penis, and by visually determining through the transparent material of the sheath where the flaccid tissue makes sufficient contact with the inside surface of the sheath, the physician can determine which diameter is appropriate for the user. Ideally, the sizers 122 are provided to measure both diameter and length so as to choose an appropriately sized collecter which provides the best fit for a particular user.

In addition, an embodiment of the sizer 122 having a constant diameter can be provided for measuring the length of the flaccid penis and choose a collector 101 of appropriate length. Again, the sizer 122 can be fabricated from a transparent material, and include markings along its length, which markings can be associated with measurements in centimeters, for example, or arbitrary markings. Such markings can be associated with a collector 101 having an associated length known to the physician fitting the user for such a device according to the invention. An alphanumeric, or other arbitrary marking allows for fabrication of collector 101 in sizes as varied as may be desired, and can also usefully determine the optimum size for an embodiment of the collector sizer 122 utilized for applying the collector 101 to the penis.

Although measuring the diameter is a more important factor for ensuring proper fit of the collector 101, establishing the proper length is also desirable. Not only is it desirable to ensure a snug and water-tight fit along the shaft, but also by minimizing dead space beyond the penile head, a closer fit reduces the head's contact with the passing urine.

A urine collection device 100, according to the invention, can be used in a number of ways. One manner of use of the device 100, described below, illustrates some of the benefits of the herein described invention.

Emptying of a urine collection device 100 according to the invention can be easily and inconspicuously performed by a user of the device in the booth of a public toilet. For example, steps for emptying the device in one manner of a method of use include: a user lowering his trousers while standing to facilitate access to the device, then detaching the reservoir 103 from the user's person, while the collector 101 can remain attached to the penis. While the collector 101 may remain attached with or without the conduit 102, preferably the collector 101 is provided to remain attached with the conduit 102. The user can then detach the connector 104 from the reservoir 104, such as by unscrewing the inlet cap, and then holding the now free end of the conduit 102 and connector 104 above the collector 101 to prevent fluid from flowing out of the conduit 102. The detaching of the connector 104 by the user is facilitated by the flexibility of the conduit 102 which can remain attached to the connector 104. The user can then sit down while holding the reservoir 103 upright in one hand, and the conduit and collector upright in the other. In a case where the user is sitting on a toilet, the user can then lower the conduit 102 and allow it to hang down into the bowl to drip empty, and the user can also empty the reservoir by pouring its contents into the toilet bowl. The user can then reconnect the connector 104 to the reservoir 103. In addition, the user can then reconnect the reservoir to the user's leg, which can be done while the stands to refasten it to the thigh.

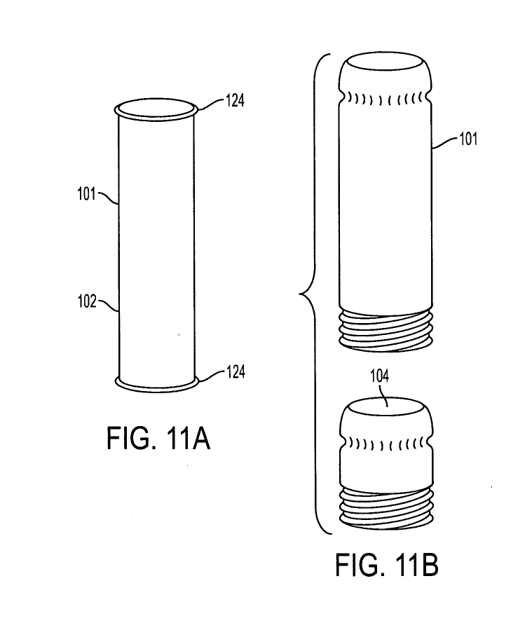

FIG. 11a shows an alternative embodiment of a collector 101 and conduit 102 according to the invention. The collector 101 and conduit 102 can be provided as a unitary device comprising a tube of uniform diameter fabricated of a flexible material, such as latex, and having a rolled portion at a proximal end of the collector 101. In addition or in the alternative, a rolled portion can be provided at the distal end of the conduit 102. Such an embodiment benefits from ease of manufacture and by facilitating the adaptation of the device to subjects of various diameters. Alternatively, as shown in FIG. 11b, the conduit 101 and connector are separate, and the conduit 101 is provided with a distal end that can be fitted to a proximal end of a connector 102 of similar or nearly similar diameter.

It can be understood by a person of ordinary skill in the art that drainage from the collector and conduit can depend on gravity, which implies a standing position. If the subject is seated, two potential problems can occur which the device addresses, namely reflux from the reservoir or accumulation in the collector and/or conduit. The former is addressed by including a check valve in the connector. Alternatively, positioning the reservoir on the thigh may reduce this problem, as illustrated in FIGS. 4C and 4E. The latter problem can cause a conduit fabricated of thin material to become distended by the accumulation of urine in the conduit, and potentially form a kink in the conduit which may block the passage or urine. This problem can be obviated by fabricating a conduit of heavier material, or by the user standing periodically, such as for a minute every half hour.

1. A urine collection system comprising
a collector, a conduit, a reservoir, a flow restrictor and at least one leg strap;
wherein said collector is connected to said conduit for passing fluid to said conduit; wherein said conduit is connected to said reservoir for passing fluid to said reservoir; wherein said leg strap is connected to said reservoir for attaching said reservoir to a leg of a user;
wherein the collector comprises a flexible material to provide a substantially water-tight fit over a user's penis;
wherein the conduit comprises a tube having a smaller diameter than the collector;
wherein the restrictor comprises a small aperture whereby fluid from the conduit can pass slowly into the reservoir, and whereby rapid backflow of fluid from the reservoir is avoided.

2. A urine collection system according to claim 1, wherein the collector and conduit are integral and fabricated from a flexible material.

3. A urine collection system according to claim 1, wherein the reservoir comprises a chamber formed from a flexible material, having an attachment point at a top end for receiving the connector, and comprising a plurality of attachment points for attachment of said leg straps to said reservoir, said attachment points comprising a plurality of loops and associated hooks.

4. A urine collection system according to claim 1, wherein said reservoir comprises a reservoir inlet that is offset from the center of a side of the reservoir and disposed proximate to the end of a top side of the reservoir; and wherein the reservoir has a curved inside face when worn by a user.

5. A urine collection system according to claim 1, further comprising a cinch disposed over the conduit for securing the conduit on the connector; and wherein the connector includes a tube passing therethrough, said tube having a notch at an outside end of the tube, said notch being disposed around at least a portion of the circumference of the tube for receiving a portion of the conduit being secured by the cinch.

6. A urine collection system according to claim 1, wherein the connector comprises a tube passing therethrough and a screw cap adapted for connection to a connection point at a top edge of the reservoir, and the flow restrictor comprises a rubber cot having a small hole at a lower end thereof, said flow restrictor being disposed over an inside end of the tube.

7. A urine collection system according to claim 1, wherein the connector comprises a tube passing therethrough and a screw cap adapted for connection to a connection point at a top edge of the reservoir, and the flow restrictor further comprises an distal restrictor having an aperture disposed at an anterior portion of the restrictor.

8. A urine collection system according to claim 7, wherein the distal restrictor comprises a swagged chamber.

9. A urine collection system according to claim 1, wherein the connector comprises a tube passing therethrough, and the flow restrictor further comprises a check valve, a ball obturator and an obturator housing.

10. A urine collection system according to claim 1, wherein the connector comprises a tube passing therethrough, and the flow restrictor further comprises a check valve, a disciform obturator and an obturator housing.

11. A urine collection system according to claim 1 wherein said collector and conduit are provided as a unitary structure having a similar diameter.

12. A urine collection system according to claim 1 wherein said collector and conduit are provided as a separate structures having a similar diameter.

13. A urine collection system comprising a collector, a conduit, a reservoir, a flow restrictor, at least one leg strap, and an application sheath;
wherein said collector is connected to said conduit for passing fluid to said conduit; wherein said conduit is connect to said reservoir for passing fluid to said reservoir; wherein said leg strap is connected to said reservoir for attaching said reservoir to a leg of a user;
wherein the collector comprises a flexible material for provided a substantially water-tight fit over a user's penis;
wherein the conduit comprises a tube having a smaller diameter than the collector;
wherein the restrictor comprises a small aperture whereby fluid from the conduit can pass slowly into the reservoir, and whereby rapid backflow of fluid from the reservoir is avoided,
and wherein the application sheath comprises two hemicylindrical shaped parts together form the shape of a tube.

14. A urine collection system according to claim 13, wherein at least one of the two hemicylindrical shaped parts further comprises a flared portion at a proximal end.

15. A urine collection system according to claim 13, wherein at least one of the two hemicylindrical shaped parts further comprises projection at a proximal end.

16. A urine collection system comprising a collector, a conduit, a reservoir, a flow restrictor, at least one leg strap, and a collector sizer;
wherein said collector is connected to said conduit for passing fluid to said conduit; wherein said conduit is connect to said reservoir for passing fluid to said reservoir; wherein said leg strap is connected to said reservoir for attaching said reservoir to a leg of a user;
wherein the collector comprises a flexible material for provided a substantially water-tight fit over a user's penis;
wherein the conduit comprises a tube having a smaller diameter than the collector;
wherein the restrictor comprises a small aperture whereby fluid from the conduit can pass slowly into the reservoir, and whereby rapid backflow of fluid from the reservoir is avoided, and
wherein the collector sizer comprises two hemicylindrical shaped parts together form the shape of a tube.

17. A urine collection system according to claim 16, wherein the collector sizer is fabricated from a transparent material.

18. A urine collection system according to claim 16, wherein the system further comprises a plurality of collector sizers having different diameters.

19. A urine collection system according to claim 16, wherein the collector sizer includes markings for measurement along its length, wherein such markings include arbitrary designations.

20. A urine collection system according to claim 16, wherein the system further comprises a plurality of collector sizers having different diameters.
